(19) United States
(12) Plant Patent Application Publication
(10) Pub. No.: US 2015/0163974 P1
(43) Pub. Date: Jun. 11, 2015

(54) Apple tree named 'Jive'

(50) ;

(71) Applicant: Edward Mark Burkitt, Hastings (NZ)

(72) Inventor: Edward Mark Burkitt, Hastings (NZ)

(21) Appl. No.: 14/544,113

(22) Filed: Nov. 24, 2014

Related U.S. Application Data
(60) Provisional application No. 61/913,043, filed on Dec. 6, 2013.

(51) Int. Cl. A01H 5/00

(52) U.S. Cl. PLT/161

(57) ABSTRACT

A new and distinct apple tree is disclosed. ‘Jive’, a limb sport of ‘GS48’, is notable for its distinctive fruit, which exhibits a red coloration, with strong defined striping present.

### CROSS-REFERENCE TO RELATED APPLICATIONS

This application claims priority under 35 USC 119(e) of prior U.S. Provisional Application Ser. No. 61/913,043, filed Dec. 6, 2013.

### STATEMENT REGARDING FEDERALLY SPONSORED RESEARCH OR DEVELOPMENT

None.

### LATIN NAME OF THE GENUS AND SPECIES OF THE PLANT CLAIMED

Malus Mill.

### VARIETY DENOMINATION

‘Jive’

### BACKGROUND OF THE INVENTION

The new apple cultivar originated as a limb sport mutation of ‘GS48’ (unpatented). It was discovered by the inventor in March 2001 in a cultivated orchard at Pakowhai, New Zealand. ‘Jive’ was asexually propagated at Pakowhai, New Zealand in 2001, and has been shown to remain true to type over successive generations.

### BRIEF SUMMARY OF THE INVENTION

‘Jive’ is a new and distinct apple tree notable for its distinctive fruit, which exhibits a red coloration.

### BRIEF DESCRIPTION OF THE PHOTOGRAPH

FIG. 1 shows the tree and fruit of the new cultivar; and

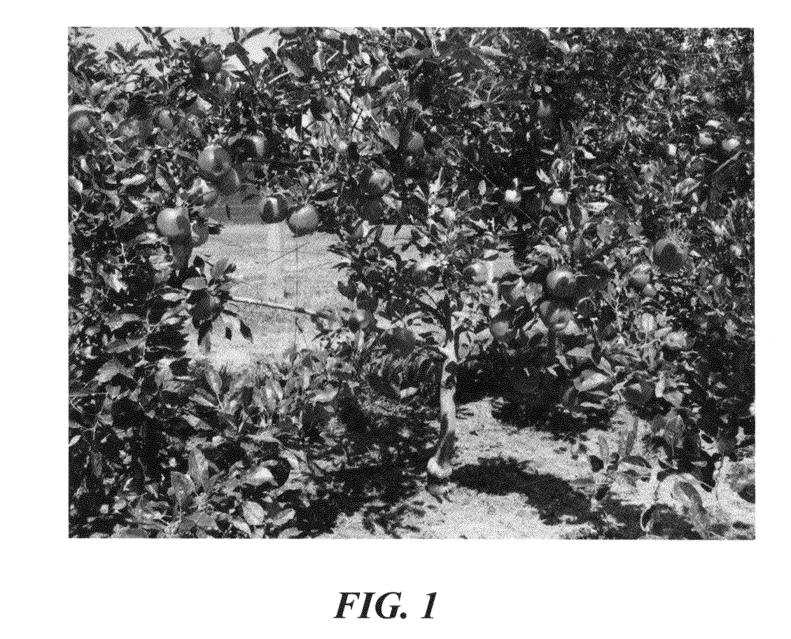

FIG. 2 shows the fruit and leaves of the new cultivar.

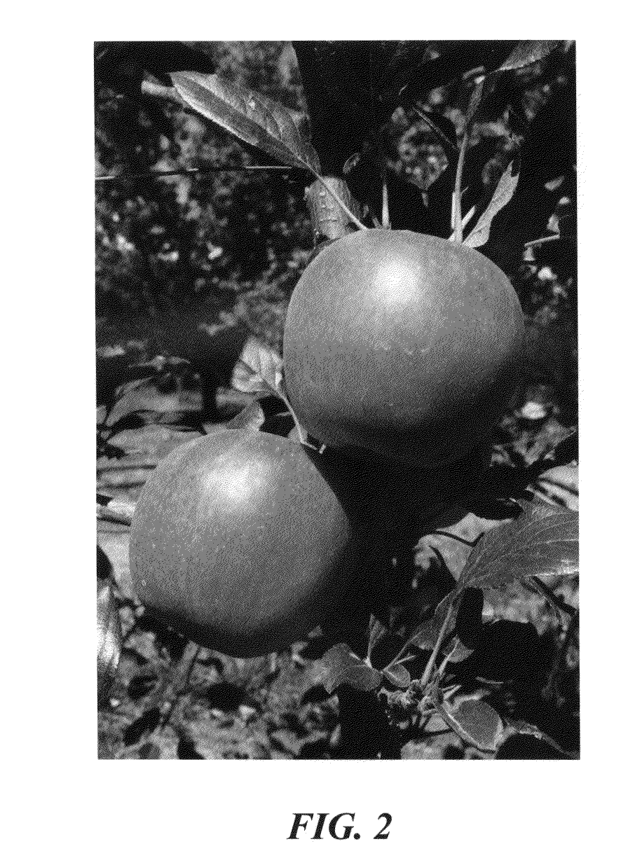

### DETAILED BOTANICAL DESCRIPTION

The following detailed botanical description is based on observations made during the 2006/2007 growing season at Pakowhai, New Zealand. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree:
   Vigor.—Medium-Strong.
   Habit.—Upright.
   Type.—Ramified.
Branch:
   Crotch angle.—Flat.
Dormant one year old shoot:
   Color.—Reddish brown.
   Pubescence.—Med-strong.
   Thickness (center diameter of middle internode).—Medium.
   Internode length.—Medium.
Winter hardiness:
   Chilling requirement.—Medium.
Flower:
   Color at balloon stage.—Light pink to dark pink.
   Diameter of open flower.—Medium.
   Size (diameter with petals pressed flat).—Medium.
   Relative position of margins.—Overlapping.
Blooming:
   Date of first bloom.—September 13^{th} in Pakowhai NZ.
   Date of full bloom.—September 22^{nd} in Pakowhai NZ.
Leaf:
   Length.—Short to Medium.
   Width.—Medium.
   Ratio: length/width.—Large.
   Shape of margins.—Serrate.
   Petiole length.—Medium to long; Average 34 mm.
Fruit:
   Size.—Large; 219 gm.
   Height.—72.6 mm.
   Diameter.—74.4 mm.
   Ratio: height/width.—Very large; 0.98.
   Shape.—Globose to obloid.
   Ribbing.—Absent.
   Crowning at calyx end.—Moderate.
   Aperture of eye.—Medium.
   Size of eye.—Medium to large.
   Length of sepal.—Medium.
   Depth of eye basin.—Deep; 11.4 mm.
   Width of eye basin.—Broad; 31.6 mm.
   Thickness of stalk.—Medium.
   Length of stalk.—Short to medium.
   Depth of stalk cavity.—Deep; 15.6 mm.
   Width of stalk cavity.—Broad; 33.8 mm.
   Greasiness of skin.—Absent or very weak.
   Ground color of skin.—Yellow to green.
   Amount of overcolor of skin.—Large.
   Color of overcolor of skin.—Orange-red.
   Intensity of overcolor.—Medium.
   Pattern of overcolor.—Solid flush with strongly defined stripes.
   Flesh texture.—Medium.
   Juiciness.—Medium to high.
   Brix.—13° to 16°.
   Flesh color.—Cream.
Harvest:
   Amount of fruit produced per tree per harvest.—Highly productive.
   Harvest date range.—Early to late March in Pakowhai, NZ.
Diseases/pests:
   Susceptibility.—Low to powdery mildew.
   Market use.—Large, red, sweet, juicy.

1. We claim a new and distinct apple tree substantially as shown and described herein.
